(19) United States
(12) Patent Application Publication
(10) Pub. No.: US 2013/0039873 A1
(43) Pub. Date: Feb. 14, 2013

(54) HAIR AND SKIN CONDITIONING COMPOSITIONS

(71) Applicant: Adam Patrick Schrott, Cincinnati, OH (US)

(72) Inventor: Adam Patrick Schrott, Cincinnati, OH (US)

(73) Assignee: Kao Corporation, Tokyo (JP)

(21) Appl. No.: 13/208,796

(22) Filed: Aug. 12, 2011

(51) Int. Cl. A61K 8/89; A61Q 19/00; A61Q 5/04; A61Q 5/06; A61K 8/81; A61Q 5/12

(52) U.S. Cl. 424/70.2; 424/70.12; 424/70.11; 424/78.03

(57) ABSTRACT

Compositions for providing hair and skin care benefits, such as smoothing, anti-static control, color protection, frizz control and moisturization are disclosed. The compositions maintain clarity, show no separation upon standing, and remain flowable liquids at room temperature. The compositions comprise a silicone or hydrocarbon component having a viscosity of from about 2,500 to about 600,000 cSt at 25° C. (such as dimethicone gum or hydrogenated polydecene), together with a carrier fluid having a viscosity less than about 100 cSt at 25° C. and a flash point greater than about 85° C. (such as caprylyl methicone, isodecyl neopentanoate, or hydrogenated polyisobutene (250)). Hair care compositions, such as shampoos and conditioners containing those combinations, as well as the method of conditioning hair utilizing those compositions is also disclosed. The compositions can be applied directly to the hair (i.e., “neat”) or via a conventional hair treatment composition, such as a shampoo or a conditioner. Skin care compositions and methods are also disclosed.

This application is related to and claims priority from U.S. Provisional Patent Application No. 61/274,829, filed Aug. 18, 2010, incorporated herein by reference.

### BACKGROUND

Non-volatile silicones and non-volatile hydrocarbons are known in the art to provide a number of consumer recognized hair and skin care benefits, such as smoothing, anti-static control, color protection, frizz control and moisturization. However, those materials are very difficult to deliver in a consumer acceptable or efficacious manner to the skin or hair in their concentrated form or when they are added to a formulation in their concentrated form.

Current technology utilizes volatile cyclic siloxanes, volatile linear siloxanes or volatile hydrocarbons as carriers for very high viscosity linear polydimethylsiloxane (PDMS) materials. These materials can have a number of undesirable attributes as carriers. Specifically, they may not be cost effective, they do not provide the best vehicle for delivery of the high viscosity PDMS (as seen by decreased performance in terms of the above-mentioned attributes), and they are currently regulated or under increased scrutiny by governmental health and environmental organizations. Current compositions utilize very high viscosity polydimethylsiloxanes in volatile compounds, such as cyclopentasiloxane, cyclomethicone; low molecular weight polydimethylsiloxanes; or low molecular weight hydrocarbons, such as isododecane or isoparaffins. While these compositions may be the best available, they are not particularly effective in delivering the benefits which can be provided by the high viscosity PDMS materials.

Thus, there is a recognized need to find a delivery system which is useful for delivering the hair and skin care benefits of high viscosity linear polydimethylsiloxane materials. That is the focus of the present invention. Specifically, the present invention provides hair and skin conditioning compositions, incorporating very high viscosity non-volatile silicone and hydrocarbon materials, which may be delivered “neat” for applications such as, but not limited to, hair serums and hair shine sprays, or may be added as a “pre-mix” to known personal care compositions such as, but not limited to, shampoos, conditioners, styling creams, lotions (for hair or skin), moisturizers (for hair or skin), hair colors, permanent wave compositions or styling gels, in order to effectively deliver the benefits which are seen with the non-volatile high viscosity PDMS and hydrocarbon materials. Additionally, the compositions of the present invention maintain clarity and show no separation upon standing, remaining a flowable liquid at room temperature.

### SUMMARY

The present invention relates to hair and skin conditioning compositions which comprise (a) a silicone or a hydrocarbon component, having a viscosity of from about 2,500 to about 600,000 cSt at 25° C.; and (b) a carrier fluid having a viscosity less than about 100 cSt at 25° C. and a flash point greater than about 85° C.

Hair conditioning and skin conditioning compositions containing those defined mixtures are also claimed. Finally, the method of utilizing those mixtures to provide hair and skin conditioning benefits when applied “neat” and from a conditioning composition are also claimed.

All ratios stated herein are “by weight”, unless otherwise specified. Further, all documents cited in this application are incorporated herein by reference.

### DETAILED DESCRIPTION

The present invention relates to hair and skin conditioning compositions which comprise a silicone or a hydrocarbon component having a viscosity of from about 2,500 to about 600,000 cSt at 25° C., together with a carrier having a viscosity less than about 100 cSt at 25° C., and a flash point greater than about 85° C. The compositions can be applied “neat” directly to hair or skin, or can be applied by conventional conditioning compositions, such as shampoos, conditioners, styling creams, lotions (for hair or for skin), moisturizers (for hair or skin), hair colors, permanent wave compositions, or styling gels. The compositions of the present invention maintain clarity, show no separation upon standing, and remain flowable liquids at room temperature. The carriers that fall outside those defined herein provide compositions that tend to show haziness, cloudiness and separation upon standing.

The first component utilized in the compositions of the present invention include high viscosity silicones and high viscosity hydrocarbons (having a viscosity of from about 2,500 to about 600,000 cSt at 25° C.) which are known to have hair and skin conditioning benefits. Examples of such materials include silicone compounds having a viscosity between about 60,000 and about 600,000 cSt; polymers of decene; polymers of isobutylene; copolymers of decene and isobutylene together; hydrogenated polymers of decene and/or isobutylene; and mixtures of the foregoing materials. These materials are well-known in the art. Specific examples of these non-volatile high viscosity materials include dimethicone gum (such as that marketed as ShinEtsu KE-76-BSR); dimethicone (100,000 cSt or 60,000 cSt) (commercially marketed as Dow Corning 200 Fluid); bis-cetearyl amodimethicone (commercially available as Silsoft AX); hydrogenated polydecene (commercially available as Exxon Mobile Pure Syn 300); hydrogenated polyisobutene (commercially available as Fancor Polyiso 1200); and polyisobutene (commercially available as Permethyl 104A, 105A, or 106A). Mixtures of the foregoing materials may also be utilized.

The second component utilized in the mixtures of the present invention include carrier fluids having a viscosity less than about 100 cSt at 25° C. and a flash point greater than about 85° C. These carriers generally have a specific gravity less than about 0.9, and a refractive index of about 1.35 or greater. Examples of these materials include mono esters containing 20 or fewer carbon atoms; linear or branched hydrocarbons containing from 12 to 20 carbon atoms; linear ethers containing 20 or fewer carbon atoms; polymers containing 12 to 20 carbon atoms formed from the polymerization of isobutylene; hydrogenated polymers containing 12 to 20 carbon atoms formed from the polymerization and hydrogenation of isobutylene; alkyl methyl siloxane fluids having fewer than 10 silicon atoms and wherein the alkyl group contains 12 or fewer carbon atoms; and mixtures of the foregoing materials. Specific examples of those materials include isodecyl neopentanoate, isodecyl isononanoate, isononyl isononanoate, tridecyl neopentanoate, hydrogenated polyisobutene (250), caprylyl methicone, dicaprylyl ether, isohexadecane, and mixtures of those materials. Mixtures of isopropyl palmitate and high molecular weight hydrocarbon blends may also be used as the carrier.

The silicone and hydrocarbon materials are generally used at levels of from about 5 to about 25%, for example from about 10% to about 20%, of the binary mixture. The carrier component of the mixture is used at levels of from about 75% to about 95%, for example from about 80% to about 90%, of the binary mixture. Specific examples of these binary mixtures are set forth in the following Tables 1(A) and (B):

TABLE 1
(A)

| | wt/wt % | | | | | | | | | | | |
|---|---|---|---|---|---|---|---|---|---|---|---|---|
| Material | 1 | 2 | 3 | 4 | 5 | 6 | 7 | 8 | 9 | 10 | 11 | 12 |
| Isodecyl Neopentanoate | 85 | 85 | 85 | — | — | — | — | — | | — | 75 | — |
| Isodecyl Isononanoate | — | — | — | 85 | — | — | — | — | — | — | — | — |
| Isononyl Isononanoate | — | — | — | — | 85 | — | — | — | — | — | — | — |
| Tridecyl Neopentanoate | — | — | — | — | — | 85 | — | — | — | — | — | — |
| Hydrogenated Polyisobutene (250) | — | — | — | — | — | — | 85 | — | — | — | — | 75 |
| Caprylyl Methicone | — | — | — | — | — | — | — | 85 | — | — | — | — |
| Dicaprylyl Ether | — | — | — | — | — | — | — | — | 85 | — | — | — |
| Isohexadecane | — | — | — | — | — | — | — | — | — | 85 | — | — |
| Isopropyl Palmitate | — | — | — | — | — | — | — | — | — | — | — | — |
| Dimethicone (100,000 cSt) | 15 | — | — | 15 | 15 | 15 | 15 | 15 | 15 | 15 | — | — |
| Dimethicone (60,000 cSt) | — | 15 | — | — | — | — | — | — | — | — | — | — |
| Dimethicone (GUM) | — | — | 15 | — | — | — | — | — | — | — | — | — |
| Bis-Cetearyl Amodimethicone | — | — | — | — | — | — | — | — | — | — | 25 | 25 |
| Hydrogenated Polydecene | — | — | — | — | — | — | — | — | — | — | — | — |
| Hydrogenated Polyisobutene | — | — | — | — | — | — | — | — | — | — | — | — |
| Polyisobutene (104A) | — | — | — | — | — | — | — | — | — | — | — | — |
| Polyisobutene (105A) | — | — | — | — | — | — | — | — | — | — | — | — |
| Polyisobutene (106A) | — | — | — | — | — | — | — | — | — | — | — | — |

TABLE 1
(B)

| | wt/wt % | | | | | | | | | |
|---|---|---|---|---|---|---|---|---|---|---|
| Material | 13 | 14 | 15 | 16 | 17 | 18 | 19 | 20 | 21 | 22 |
| Isodecyl Neopentanoate | — | — | — | 85 | — | 85 | — | 85 | 85 | 85 |
| Isodecyl Isononanoate | — | — | — | — | — | — | — | — | — | — |
| Isononyl Isononanoate | — | — | — | — | — | — | — | — | — | — |
| Tridecyl Neopentanoate | — | — | — | — | — | — | — | — | — | — |
| Hydrogenated | — | — | — | — | — | — | — | — | — | — |
| Polyisobutene (250) | | | | | | | | | | |
| Caprylyl Methicone | 75 | — | — | — | — | — | — | — | — | — |
| Dicaprylyl Ether | — | 75 | — | — | — | — | — | — | — | — |
| Isohexadecane | — | — | 75 | — | — | — | — | — | — | — |
| Isopropyl Palmitate | — | — | — | — | 85 | — | 85 | — | — | — |
| Dimethicone | — | — | — | — | — | — | — | — | — | — |
| (100,000 cSt) | | | | | | | | | | |
| Dimethicone | — | — | — | — | — | — | — | — | — | — |
| (60,000 cSt) | | | | | | | | | | |
| Dimethicone | — | — | — | — | — | — | — | — | — | — |
| (GUM) | | | | | | | | | | |
| Bis-Cetearyl | 25 | 25 | 25 | — | — | — | — | — | — | — |
| Amodimethicone | | | | | | | | | | |
| Hydrogenated | — | — | — | 15 | 15 | — | — | — | — | — |
| Polydecene | | | | | | | | | | |
| Hydrogenated | — | — | — | — | — | 15 | 15 | — | — | — |
| Polyisobutene | | | | | | | | | | |
| Polyisobutene (104A) | — | — | — | — | — | — | — | 15 | — | — |
| Polyisobutene (105A) | — | — | — | — | — | — | — | — | 15 | — |
| Polyisobutene (106A) | — | — | — | — | — | — | — | — | — | 15 |

The binary mixture compositions may be applied directly to hair and skin (i.e., “neat” application) such as from a hair serum or a hair shine spray, or they may be applied from known hair and skin conditioning compositions. Such compositions include, but are not limited to, shampoos, conditioners, styling creams, lotions, moisturizers, hair coloring compositions, permanent wave compositions and styling gels. They are well-known to those skilled in the art; for example, shampoo compositions are described in U.S. Pat. No. 6,706,258, Gallagher et al, issued Mar. 16, 2004, incorporated herein by reference.

Such shampoos comprise one or more cleansing surfactants which are cosmetically acceptable and suitable for topical application to the hair. Suitable surfactants, which may be used singularly or in combination, are selected from anionic, amphoteric and zwitterionic surfactants, and mixtures thereof. Examples of anionic surfactants include alkyl sulfates, alkyl ether sulfates, alkaryl sulfonates, alkanoyl isethionates, alkyl succinates, alkyl sulfosuccinates, n-alkyl sarcosinates, alkyl phosphates, alkyl ether phosphates, alkyl ether carboxylates, and alpha-olefin sulfonates, especially their sodium, magnesium, ammonium and mono-, di- and triethanolamine salts. The alkyl and acyl groups generally contain from 8 to 18 carbon atoms and may be unsaturated. The alkyl ether sulfates, alkyl ether phosphates and alkyl ether carboxylates may contain from 1 to 10 ethylene oxide or propylene oxide units per molecule.

Typical anionic surfactants for use in shampoos include sodium oleyl succinate, ammonium lauryl sulfosuccinate, ammonium lauryl sulfate, sodium dodecylbenzene sulfonate, triethanolamine dodecyl benzene sulfonate, sodium cocoyl isethionate, sodium lauryl isethionate and sodium n-lauroyl sacrocinate. The most preferred anionic surfactants are sodium lauroyl sulfate, triethanolamine mono lauroyl phosphate, sodium lauroyl ether sulfate (1 EO, 2 EO and 3 EO), ammonium lauroyl sulfate and ammonium lauroyl ether sulfate (1 EO, 2 EO, and 3 EO).

Examples of amphoteric and zwitterionic surfactants include alkyl amine oxides, alkyl betaines, alkyl amidopropylbetaines, alkyl sulfobetaines (sultaines), alkyl glycinates, alkyl carboxy glycinates, alkyl amphopropionates, alkyl amphoglycinates, alkyl amidopropyl hydroxyl sultaines, acyl taurates, and acyl glutamates, wherein the alkyl and acyl groups contain from 8 to 19 carbon atoms. Typical amphoteric and zwitterionic surfactants for use in shampoos include lauryl amine oxide, cocodimethyl amine oxide, sulfopropyl betaine, lauryl betaine, cocamidopropyl betaine and sodium cocaminopropionate.

The shampoo compositions may additionally contain cationic deposition enhancing polymers, non-silicone conditioning agents, anti-microbial agents, and aesthetic agents, such as colorants, fragrances, and pearlescing agents. Other components which may be included include viscosity modifiers, preservatives, polyols (such as glycerin and polypropylene glycol), chelating agents (such as EDTA), antioxidants, sunscreens, and carriers (which can be, for example, ethanol and/or water).

Hair conditioning compositions are also well-known in the art. For example, they are described in U.S. Pat. No. 6,287,545, Su, issued Sep. 11, 2001, incorporated herein by reference. Such hair conditioner compositions commonly include long chain mono-, di- or even tri-higher alkyl quaternary ammonium compounds, such cetyl trimonium chloride, fatty alcohols (such as cetyl alcohol or stearyl alcohol), mineral oil, humectants, surfactants/emulsifiers, thickeners (such as cellulose derivatives), polycationic polymers (known as polyquaterniums), colorants, perfumes, opacifiers, pearlescent aides, buffers, preservatives, antioxidants, and carriers (which can be, for example, ethanol and/or water).

Examples of conditioner formulations, in general terms, which include the binary compositions of the present invention are set forth in the following Table 2:

TABLE 2
General Examples of Conditioner Formulations

| General Material Description | General wt/wt % |
|---|---|
| Carrier | Balance |
| Choose from Lactic, Malic, | 0.2-0.6 |
| Tartaric, Fumaric, Isononanoic Acids | |
| C16-18 Fatty Alcohol | 4.0-6.0 |
| Ester | 0.2-0.4 |
| Alkylamidoamine | 1.0-2.0 |
| “Neat” Formulation #X | 0.75-3.0 |
| (see Tables 1A and 1B) | |
| Preservative | as appropriate |
| Fragrance | as appropriate |

Examples of other types of hair care products, including hair styling products, are taught, for example, in U.S. Pat. No. 7,850,952, Ivanova et al, issued Dec. 14, 2010; U.S. Pat. No. 7,785,575, Anderson et al, issued Aug. 31, 2010; U.S. Pat. No. 6,586,378, Chandra, issued Jul. 1, 2003; and U.S. Pat. No. 6,136,296, Midha et al, issued Oct. 24, 2000, all of which are incorporated herein by reference.

The compositions described can also be used to provide moisturization benefits to skin. Skin care compositions can include any of the composition types known in the art, such as, for example, lotions. Examples of skin care lotions are taught in U.S. Pat. No. 7,816,310, Landa et al, issued Oct. 19, 2010, incorporated herein by reference.

When utilized as described herein, the compositions of the present invention are applied in an “effective amount”, i.e., an amount which provides the hair or skin conditioning and/or moisturizing benefits desired, but not so much as to result in undesirable effects to the user (e.g., greasiness to the hair or skin).

Examples of compositions of the present invention are set forth below. They are intended to be merely illustrative, and not limiting, of the present invention.

### Examples 1-8

Conditioner formulations incorporating the binary mixtures of the present invention contain the following components and are prepared using conventional techniques:

| | | Weight % | | | |
|---|---|---|---|---|---|
| Conditioner Formulations | | 1 | 2 | 3 | 4 |
| A | pH Regulator (Lactic Acid, | qs | qs | qs | qs |
| | Malic Acid, Tartaric Acid, | | | | |
| | Fumaric Acid, Isononanoic | | | | |
| | Acid) | | | | |
| | Glycerin | | | | 12.00 |
| B | Cetearyl Alcohol | 5.00 | 5.00 | 5.00 | 5.00 |
| | Cetyl Esters | 1.00 | 1.00 | 1.00 | 1.00 |
| | Behenamidopropyl | 1.50 | 1.50 | 1.50 | 1.50 |
| | Dimethylamine | | | | |
| | Distearyldimonium Chloride | | 0.15 | | |
| | Behentrimonium Chloride | | | | |
| C | Blend #1 | 3.00 | | | |
| | (15% 100,000 cSt | | | | |
| | Dimethicone + 85% | | | | |
| | Isodecyl Neopentanoate) | | | | |
| | Blend #9 | | 2.00 | | 2.00 |
| | (15% 100,000 cSt | | | | |
| | Dimethicone + 85% | | | | |
| | Isononyl Isononanoate) | | | | |
| | Blend #11 | | | 1.25 | |
| | (25% Bis-Cetearyl | | | | |
| | Amodimethicone + 75% | | | | |
| | Isodecyl Neopentanoate) | | | | |
| D | Preservative (Choice) | qs | qs | qs | qs |
| | Fragrance | qs | qs | qs | qs |
| | Water | Balance | Balance | Balance | Balance |

| | |
|---|---|
| Target pH | 3.5-5.0 |

| | | Weight % | | | |
|---|---|---|---|---|---|
| Conditioner Formulations | | 5 | 6 | 7 | 8 |
| A | pH Regulator (Lactic Acid, | qs | qs | qs. | qs. |
| | Malic Acid, Tartaric Acid, | | | | |
| | Fumaric Acid, Isononanoic | | | | |
| | Acid) | | | | |
| | Glycerin | | | | 8.00 |
| B | Cetearyl Alcohol | 5.00 | 5.00 | 5.00 | 5.00 |
| | Cetyl Esters | 1.50 | 1.50 | 1.50 | 1.50 |
| | Behenamidopropyl | | | | |
| | Dimethylamine | | | | |
| | Distearyldimonium Chloride | | 0.15 | | |
| | Behentrimonium Chloride | 3.00 | 3.00 | 3.00 | 3.00 |
| C | Blend #1 | 2.50 | | | 3.50 |
| | (15% 100,000 cSt | | | | |
| | Dimethicone + 85% | | | | |
| | Isodecyl Neopentanoate) | | | | |
| | Blend #9 | | 1.50 | | |
| | (15% 100,000 cSt | | | | |
| | Dimethicone + 85% | | | | |
| | Isononyl Isononanoate) | | | | |
| | Blend #11 | | | 1.00 | |
| | (25% Bis-Cetearyl | | | | |
| | Amodimethicone + 75% | | | | |
| | Isodecyl Neopentanoate) | | | | |
| D | Preservative (Choice) | qs | qs | qs | qs |
| | Fragrance | qs | qs | qs | qs |
| | Water | Balance | Balance | Balance | Balance |

| | |
|---|---|
| Target pH | 3.5-5.0 |

The compositions are made by conventional means. For example, the compositions are made as follows. In the main vessel, purified water is heated to 80° C. Add components (A) which include water-soluble ingredients (such as pH regulators, salts, glycerin). Mix until homogeneous. Components (B) comprise the “oil phase” and should be pre-mixed in a separate vessel, heated to 80° C., and mixed until homogeneous. Add the oil phase components to the main vessel with mixing to form an emulsion. The resulting emulsion may then be cooled to 40° C. Components (C) should be pre-mixed in a separate vessel until homogeneous. At 40° C., components (C) may be added with mixing to incorporate them into the emulsion. Once components (C) are completely incorporated, components (D) may be added with mixing to incorporate them into the emulsion.

When applied to hair, the conditioners provide excellent conditioning and frizz control characteristics.

In one embodiment, the silicone/hydrocarbon and the carrier fluid components, defined herein, are pre-mixed prior to mixing with other components. This pre-mixing can provide performance and formulational benefits.

### Examples 9-10

Lotion formulations of the present invention contain the components listed in the following table and are made using the procedure described above. When applied to skin, the lotions provide excellent skin conditioning and moisturizing.

| | | Weight % | |
|---|---|---|---|
| Lotion Formulation | | 9 | 10 |
| A | Glycerin | 7.50 | 7.50 |
| | pH Regulator (Lactic Acid, Malic Acid, | qs | qs |
| | Tartaric Acid, Fumaric Acid, Isononanoic Acid, | | |
| | Sodium Hydroxide) | | |
| | Carbomer | | 0.05 |
| B | Behentrimonium Chloride | 2.00 | |
| | Cetearyl Alcohol | 3.00 | 4.00 |
| | Petrolatum | 2.00 | 1.00 |
| | Mineral Oil | 1.25 | 1.50 |
| | Ceteareth-20 | | 1.00 |
| C | Blend #19 | 4.00 | |
| | (15% Hydrogenated Polyisobutene + 85% | | |
| | Isopropyl Palmitate) | | |
| | Blend #8 | | 3.00 |
| | (15% 100,000 cSt Dimethicone + 85% | | |
| | Isononyl Isononanoate) | | |
| | Blend #11 | | 1.00 |
| | (15% 100,000 cSt Dimethicone + 85% | | |
| | Caprylyl Methicone) | | |
| D | Preservative (Choice) | qs | qs |
| | Fragrance | qs | qs |
| | Water | Balance | Balance |

What is claimed is:

1. A hair and skin conditioning composition comprising (a) a silicone or hydrocarbon component, having a viscosity of from about 2,500 to about 600,000 cSt at 25° C.; and (b) a carrier fluid having a viscosity less than about 100 cSt at 25° C. and a flash point greater than about 85° C.

2. The composition according to claim 1 wherein components (a) and (b) are premixed prior to their mixture with the remainder of the composition.

3. The composition according to claim 1 wherein component (b) has a specific gravity less than about 0.9 and a refractive index of about 1.35 or greater.

4. The composition according to claim 1 wherein component (a) is present at from about 5% to 25% of the composition and component (b) is present at from about 75% to about 95% of the composition.

5. The composition according to claim 4 wherein component (a) is present at from about 10% to about 20% of the composition, and component (b) is present at from 80% to about 90% of the composition.

6. The composition according to claim 4 wherein component (a) is selected from the group consisting of (i) silicone compounds having a viscosity between about 60,000 and about 600,000 cSt; (ii) polymers of decene; (iii) polymers of isobutylene; (iv) polymers of decene and isobutylene; (v) hydrogenated polymers of decene and/or isobutylene; and (vi) mixtures thereof.

7. The composition according to claim 4 wherein component (b) is selected from the group consisting of (i) mono esters containing 20 or fewer carbon atoms; (ii) linear or branched hydrocarbons containing from 12 to 20 carbon atoms; (iii) linear ethers containing 20 or fewer carbon atoms; (iv) polymers containing from 12 to 20 or carbon atoms formed from the polymerization of isobutylene; (v) hydrogenated polymers having containing 12 to 20 or carbon atoms formed from the polymerization and hydrogenation of isobutylene; (vi) alkyl methyl siloxane fluids having fewer than 10 silicon atoms and wherein the alkyl group contains 12 or fewer carbon atoms; and (vii) mixtures thereof.

8. The composition according to claim 6 wherein component (b) is selected from the group consisting of (i) mono esters containing from 12 to 20 carbon atoms; (ii) linear or branched hydrocarbons containing 20 or fewer carbon atoms; (iii) linear ethers containing 20 or fewer carbon atoms; (iv) polymers containing from 12 to 20 carbon atoms formed from the polymerization of isobutylene; (v) hydrogenated polymers containing from 12 to 20 carbon atoms formed from the polymerization and hydrogenation of isobutylene; (vi) alkyl methyl siloxane fluids having fewer than 10 silicon atoms and wherein the alkyl group contains 12 or fewer carbon atoms; and (vii) mixtures thereof.

9. The composition according to claim 8 wherein component (a) is selected from the group consisting of dimethicone gum, dimethicone (100,000 cSt), dimethicone (60,000 cSt), bis-cetearyl amodimethicone, hydrogenated polydecene, hydrogenated polyisobutene, polyisobutene, and mixtures thereof; and component (b) is selected from the group consisting of isodecyl neopentanoate, isodecyl isononanoate, isononyl isononanoate, tridecyl neopenatanoate, hydrogenated polyisobutene (250), caprylyl methicone, diceprylyl ether, isohexadecane, and mixtures thereof.

10. A hair conditioning composition selected from shampoos, conditioners, styling creams, styling lotions, moisturizers, coloring compositions, permanent wave compositions, and styling gels which comprise from about 0.5% to about 5% of the composition according to claim 4.

11. A hair styling composition selected from shampoos, conditioners, styling creams, styling lotions, moisturizers, coloring compositions, permanent wave compositions, and styling gels, which comprise from about 0.5% to about 5% of the composition according to claim 9.

12. A method of styling and conditioning hair by applying to said hair an effective amount of the composition according to claim 4.

13. A method of styling and conditioning hair by applying to said hair an effective amount of the composition according to claim 9.

14. A method of styling and conditioning hair by applying to said hair an effective amount of the composition according to claim 11.

15. A method of styling and conditioning hair by applying to said hair an effective amount of the composition according to claim 4.

16. A skin conditioning composition in the form of a lotion comprising from about 0.5% to about 5% of the composition according to claim 4.

17. A method of conditioning skin by applying to said skin an effective amount of the composition according to claim 4.

18. The composition of claim 10 which includes a vehicle which comprises water.

19. The composition of claim 11 which includes a vehicle which comprises water.
